(19) United States
(12) Patent Application Publication
(10) Pub. No.: US 2009/0275965 A1
(43) Pub. Date: Nov. 5, 2009

(54) SURGICAL INSTRUMENT

(76) Inventors: Paul Sibbons, Middlesex (GB); Richard Cohen, London (GB)

Correspondence Address: STETINA BRUNDA GARRED & BRUCKER, 75 ENTERPRISE, SUITE 250, ALISO VIEJO, CA, 92656, US

(21) Appl. No.: 12/158,625

(22) PCT Filed: Dec. 21, 2006
(86) PCT No.: PCT/GB2006/004899

§ 371 Date: Oct. 29, 2008

(30) Foreign Application Priority Data
Dec. 21, 2005 (GB) 0525930.4

(51) Int. Cl. A61B 17/32

(52) U.S. Cl. 606/170

(57) ABSTRACT

A surgical device comprising a head with a cutting edge and a shaft is described for use in treating anal fistulas. The device facilitates the removal of tissue from the walls of the fistula allowing the introduction of collagen that aids the healing of the fistula. In this way a fistula may be closed without serious consequences to the continence of the patient.

This invention relates to a surgical device for removing granulation tissue. The invention is applicable for use when treating anal fistulas.

### BACKGROUND OF THE INVENTION

A fistula is a narrow conduit that develops between the internal part of the rectum or colon and the external skin around the anus. The conduit allows the passage of small amounts of the content of the rectum or colon to the external opening in the skin around the anus. This passage of the contents of the bowel through tissue is painful and can be embarrassing to the afflicted person as well as encouraging infection in the tissue lining the fistula.

There are two possible treatments for a fistula. The most common treatment is the placement of a length of surgical suture through the fistula that is then tied to produce a loop, known as a seton, which remains in place through the fistula to encourage drainage of the fistula and thereby decreasing the incidence of infection within the fistula.

The other treatment involves cutting through the anal skin to the fistula. This allows the fistula to be opened up along its length and for the granulation tissue that has built up on the internal surfaces of the fistula to be removed. Once the granulation tissue has been removed the fistula can then be closed and then in the best case scenario the fistula will heal up. However, this is not always the case.

### SUMMARY OF THE INVENTION

According to a first aspect of the invention there is provided a device adapted to remove tissue from a fistula, the device comprising an elongated shaft, a head attached to one end of the shaft, the head being provided with a cutting edge.

Preferably the head is a tubular head which may be closed at the aspect furthest from the shaft. Preferably the cutting edge is a serrated cutting edge in order to facilitate removal of tissue within the fistula.

The device may also be provided with an attachment point adapted to attach to a seton at the other end to the head. This facilitates movement of the device through the fistula. The attachment point may also be adapted to attach to a high-speed rotary device to aid removal of tissue within the fistula.

Preferably the shaft is hollow and provided with an input connected to a fluid source and a fluid output for emitting fluid into the fistula. The fluid output may be in the head and the fluid may be input to the shaft via the attachment point.

According to a first aspect of the invention there is provided a method of treating a fistula comprising the steps of removing the granulation tissue from the surface of the fistula, inserting acellular porcine dermal collagen into the fistula and sealing the openings of the fistula once the fistula is filled with the collagen.

### BRIEF DESCRIPTION OF THE DRAWINGS

Other aspects and features of the present invention will become apparent to those ordinarily skilled in the art upon review of the following description of specific embodiments of the invention in conjunction with the accompanying figures.

FIG. 1 illustrates the surgical device of the present invention; and

FIG. 2 illustrates the surgical device of the present invention adapted to allow fluid to flow through the device.

### DETAILED DESCRIPTION OF PREFERRED EMBODIMENTS

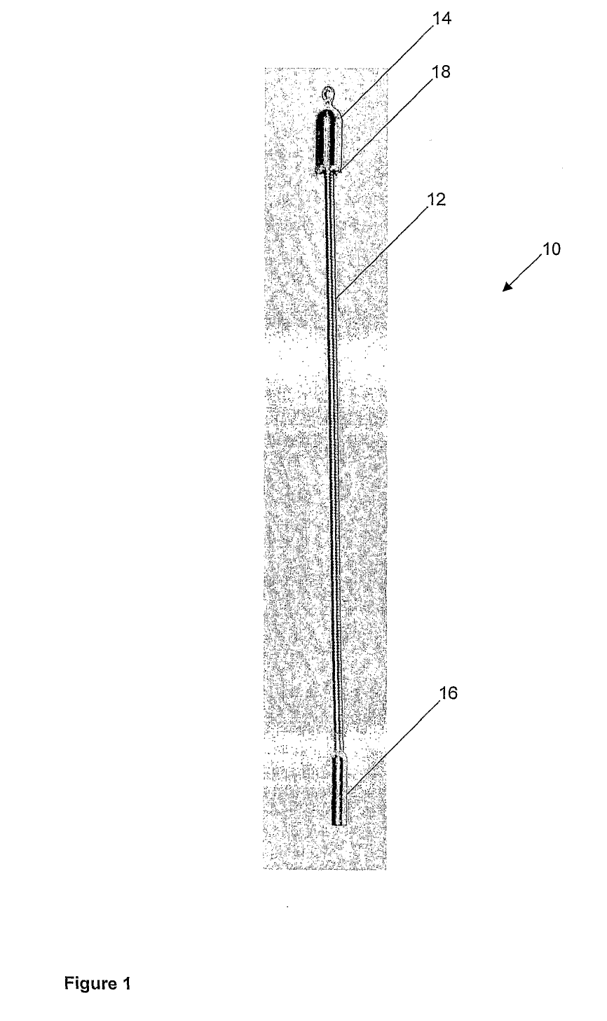

With reference to FIG. 1, the surgical device 10 is designed to remove the granulation tissue from the fistula without having to open the fistula along its length. In order to achieve this it has a shaft 12 with a head 14 mounted at one end and an attachment point 16 at the other.

The tubular head 14 is provided with a cutting edge 18 on the end closest to the shaft 12. The cutting edge 18 is situated such that when the device is pulled through the fistula using the shaft 12 it removes any granulation tissue that has built up on the surface of the fistula. Preferably the cutting edge 18 is serrated in order to improve the removal of granulation tissue.

Additionally, it is preferable that the head 14 is cylindrical in order to minimise any additional damage to the fistula's surface. The tubular head 14 may have a closed aspect at the end of the head furthest from the cutting edge 18. This prevents granulation tissue that has been removed from the surface of the fistula from migrating back up towards the internal opening of the fistula. In this way the cut surface of the fistula is kept clean.

At a second end of the shaft 12 there is provided an attachment point 16. The attachment point 16 may serve to allow the device 10 to be attached to a seton in order to aid the device's passage through the fistula. The attachment point 14 may also allow the device 10 to be attached to a high-speed rotation source, such as an orthopaedic drill. The rotation of the device provided by the rotation source improves the removal of the granulation tissue by the cutting edge 18 within the fistula.

A method of using the device 10 will now be described. The fistula, which is to be treated, is provided with a seton, the loop of suture as described above. The surgical device 10 is then inserted through the anus, with the head end 14 being inserted first, until the attachment point 16 reaches the inner opening of the fistula. The attachment point 16 is then attached to the seton and the seton is used to move the device 10 through the fistula until the end of the shaft 12 protrudes from the external opening of the fistula.

The seton is removed and the device 10 is pulled through the fistula. As described above the head 14 is provided with a cutting edge 18. When the device 10 is pulled through the fistula the cutting edge 18 acts to remove the granulation layer that has formed on the surface of the fistula. This process may be repeated if not all the granulation material is removed in the first instance.

As described above the attachment point 16 on the shaft may optionally be adapted to allow a high-speed rotation device to be attached to the device 10. The rotation device is preferably attached to the device 10 when the attachment point 16 first protrudes from the fistula but rotation is only applied when the head 14 is adjacent to the inner opening of the fistula.

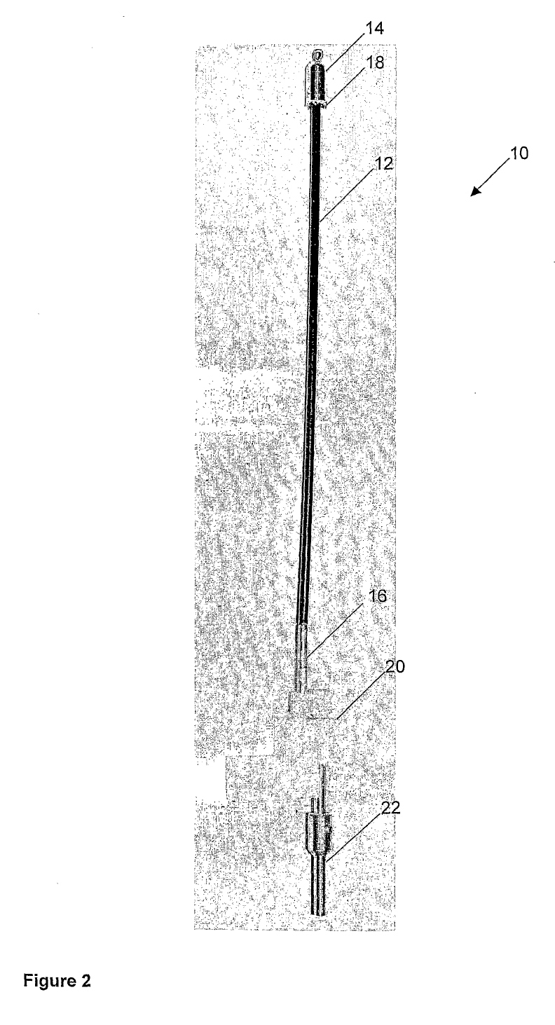

In an alternative embodiment of the present invention the shaft 12 is a hollow shaft and is provided with an entry point for fluid at the opposite end to the head. The head 14 is provided with one or more exit points for the fluid. The fluid entry point is connected to a fluid source, for example using an attachment 20 as shown in FIG. 2. The attachment 20 has an attachment point 22 enabling a rotation device to be attached to the surgical device 10 that preferably supplies fluid at high pressures to the device 10. The fluid may be any solution suitable for flushing out fistulas to remove the granulation tissue cut off by the cutting surface.

Once the granulation tissue has been removed the device 10 is also removed from the fistula and a catheter, for example a 14 gauge catheter, is inserted into the fistula from the, external opening. Preferably the catheter is inserted until the tip of the catheter protrudes from the internal opening. A suture is loosely applied to the mucosal lining at the internal opening of the fistula. Acellular porcine dermal collagen such as Permacol® is then injected into the opening until there is an excess and the suture is then tied close to the internal opening. The collagen is then injected to fill the fistula with the catheter gradually being withdrawn from the fistula. When the fistula is full of the collagen the catheter is removed and the external opening of the fistula is closed with a suture.

In this way the fistula is able to heal without any further contents of the digestive tract passing through it.

1-12. (canceled)

13. A device adapted to remove tissue from a fistula, the device comprising:
(a) an elongated shaft
(b) a head attached to one end of the shaft; the head being provided with a serrated cutting edge configured to remove a layer of granulation tissue from a fistula.

14. A device as claimed in claim 13 wherein the head is a tubular head.

16. A device as claimed in claim 13 wherein the head is closed at the end furthest from the shaft.

17. A device as claimed in claim 13 wherein an attachment point adapted to attach to a seton is provided at the other end to the head.

18. A device as claimed in claim 17 wherein the attachment point is further adapted to attach to a high-speed rotary device.

19. A device as claimed in claim 13 wherein the shaft is hollow and provided with an input connected to a fluid source and a fluid output for emitting fluid into the fistula.

20. A device as claimed in claim 19 wherein the fluid output is in the head.

21. A device as claimed in claim 19 wherein the fluid is input to the shaft via the attachment point.

22. A method of treating a fistula comprising the steps of:
(a) removing the granulation tissue from the surface of the fistula;
(b) inserting acellular porcine dermal collagen into the fistula; and
(c) sealing the openings of the fistula once the fistula is filled with the collagen.
